(19) United States
(12) Patent Application Publication
(10) Pub. No.: US 2025/0329422 A1
(43) Pub. Date: Oct. 23, 2025

(54) METHOD FOR QUANTIFYING OVERLAPPING CHEMICAL SIGNALS

(71) Applicant: The United States of America, as represented by the Secretary of Commerce, Silver Spring, MD (US)

(72) Inventor: Stephen Alfred Montzka, Boulder, CO (US)

(21) Appl. No.: 19/180,389

(22) Filed: Apr. 16, 2025

Related U.S. Application Data
(60) Provisional application No. 63/636,382, filed on Apr. 19, 2024.

(51) Int. Cl. G16C 20/20; G01N 30/86

(52) U.S. Cl. CPC G16C 20/20; G01N 30/8631; G01N 30/8696

(57) ABSTRACT

Embodiments in accordance with the present invention relate to methods for quantifying overlapping chemical signals in environmental analysis without any substantial modification of instrumental analysis parameters. Methods in accordance with embodiments of the present invention are capable of resolving overlapping chemical signals (peaks) in chromatographic analysis when it is not possible to realize both normal conditions for separation: in time as the chemicals elute from the chromatographic column and by mass fragment ions created in the mass spectrometer. A method in accordance with embodiments of the present invention involves the use of component response factors to achieve quantification.

### CROSS-REFERENCE TO RELATED APPLICATION

This application claims the benefit of priority from U.S. Provisional Patent Application Ser. No. 63/636,382, filed on Apr. 19, 2024, the disclosure of which is incorporated herein by reference in its entirety.

### STATEMENT REGARDING FEDERAL RIGHTS

The invention described herein was made with support from the National Oceanic and Atmospheric Administration (NOAA) of the United States Department of Commerce. The United States Government has certain rights in the invention.

### FIELD OF THE INVENTION

The present invention relates generally to methods for analysis of chemical samples, and more particularly, to methods for analysis of chemicals in environmental samples.

### BACKGROUND OF THE INVENTION

Some estimates of global CFC-113 (CCl₂FCClF₂) emissions derived from measured atmospheric concentrations suggest increases, potentially indicating resumed uses in controlled applications. Atmospheric measurements of CFC-113 may be biased by co-elution of its rapidly increasing isomer CCl₃CF₃ (CFC-113a), and those biases can be significant and are likely instrument-dependent. Measurements of CFC-113 and other ethane-based CFCs are prone to interferences from isomers in conventional analytical chromatographic instrumentation, such as gas chromatography coupled to electron capture or mass spectrometry detection. Interference-free quantification for any two chemicals typically requires chromatographic separation in time or, lacking that, selective detection by electron capture or mass spectrometry at unique ions. If those conditions are not met, substantial changes in the analytical method must be considered. There is a need to routinely separately quantify ethane-based CFC isomers in the chemical analysis of air samples to provide unbiased measurements of each, and provide ongoing measures of atmospheric concentration trends, distributions and co-variations. In particular, there is a need for a method to provide independent results for CFC-113 and CFC-113a. Capillary gas chromatography with mass spectrometry detection often provides a high degree of sensitivity and selectivity in the chemical compositional analysis of air, but it is not always sufficient to provide independent quantification, particularly of isomers that are difficult to separate chromatographically. When unique ions of sufficient abundance are not available to provide selectivity in the analysis, methods for deconvolving overlapping signals (peaks) that rely on some degree of separation between the two chemicals can be used. If sufficient, this separation can allow for a deconvolution of the overlapping signals with some assumptions about the peak shapes associated with each component and scaling of the peak sizes to derive the relative amounts of each component. Accordingly, there is a need for improved methods for detection and quantification of overlapping signals (peaks) obtained in chromatographic analysis that requires no assumptions about the component peak shapes and can be implemented even if there is essentially no separation in elution time of the two chemicals and when the relative abundances of the chemicals are very different.

### SUMMARY OF THE INVENTION

Embodiments in accordance with the present invention relate to methods for quantifying overlapping chemical signals in environmental analysis without any substantial modification of instrumental analysis parameters. Methods in accordance with embodiments of the present invention are capable of resolving overlapping chemical signals (peaks) in chromatographic analysis when it is not possible to realize both normal conditions for separation: in time as the chemicals clute from the chromatographic column and by mass fragment ions created in the mass spectrometer. A method in accordance with embodiments of the present invention involves the use of component response factors to achieve quantification. An advantage of the method in accordance with embodiments of the present invention includes achieving unbiased quantification of the two components without requiring modification of the chromatographic analysis parameters, which ensures that separation can be achieved with minimal disruption of typical methods for conventional chromatographic analysis.

Embodiments of the present invention relate to a method for quantifying overlapping measurement signals from analysis of a chemical sample, the method including receiving at a processor a plurality of spectra for a first chemical component and a second chemical component of the chemical sample, wherein the first and the second chemical components coelute from a chromatographic column; receiving at the processor a plurality of reference spectra for the first chemical component and the second chemical component; determining from the plurality of the reference spectra for the first chemical component and the second chemical component a plurality of first fragment ion signals for a plurality of first chemical fragment ions and a plurality of second fragment ion signals for a plurality of second chemical fragment ions; determining whether the plurality of the first fragment ion signals for the plurality of the first chemical fragment ions are different from the plurality of the second fragment ion signals for the plurality of the second chemical fragment ion; selecting from the plurality of the reference spectra for the first chemical component and the second chemical component at least one of the plurality of the first fragment ion signals for at least one of the plurality of the first chemical fragment ions and at least one of the plurality of the second fragment ion signals for the at least one of the plurality of the second chemical fragment ions when the plurality of the first fragment ion signals for the plurality of the first chemical fragment ions are not different from the plurality of the second fragment ion signals for the plurality of the second chemical fragment ions, wherein a first molar response for the first chemical component relative to the second chemical component at the at least one of the plurality of the first chemical fragment ions is different from a second molar response for the first chemical component relative to the second chemical component at the at least one of the plurality of the second chemical fragment ions; determining a plurality of first responses for the at least one of the plurality of the first chemical fragment ions per mole of the first chemical component injected into the chromatographic column and a plurality of second responses for the at least one of the plurality of the second chemical fragment ions per mole of the second chemical component injected into the chromatographic column; and determining a first measurement signal peak response corresponding to the plurality of the first responses for the at least one of the plurality of the first chemical fragment ions per mole of the first chemical component and a second measurement signal peak response corresponding to the plurality of the second responses for the at least one of the plurality of the first chemical fragment ions per mole of the first chemical component. More particularly the first chemical component is CFC-113 and the second chemical component is CFC-113a. In one embodiment, cach of the plurality of the spectra comprises a scan number, response and mass-to-charge ratio of charged particles.

In some embodiment, the method for quantifying overlapping measurement signals from analysis of a chemical sample further includes the step of determining a first relative abundance of the plurality of the first fragment ions from the plurality of the first fragment ion signals and a second relative abundance of the plurality of the second fragment ions from plurality of the second fragment ion signals. In some embodiments, the selecting from the plurality of the reference spectra for the first chemical component and the second chemical component at least one of the plurality of the first fragment ion signals for the at least one of the plurality of the first chemical fragment ions and at least one of the plurality of the second fragment ion signals for the at least one of the plurality of the second chemical fragment ions when the first relative abundance of the plurality of the first chemical fragment ions and the second relative abundance of the plurality of the second chemical fragment ions are not sufficient for the quantification of the overlapping measurement signals at a predetermined precision. In another embodiment, the selecting the at least one of the plurality of the first fragment ion signals and the at least one of the plurality of the second fragment ion signals comprises determining the first chemical fragment ion created by the first chemical component in relative abundance to the second chemical component and the second chemical fragment ion created by the second chemical component in relative abundance to the first chemical component.

In other embodiments, the first molar response for the first chemical component relative to the second chemical component at the at least one of the plurality of the first chemical fragment ions is greater than the second molar response for the first chemical component relative to the second chemical component at the at least one of the plurality of the second chemical fragment ions.

In another embodiment, the first chemical component is CFC-113 and the second chemical component is CFC-113a, wherein the first molar response for CFC-113 relative to CFC-113a at ion 103 is about 5:1, and wherein the second molar response of CFC-113 relative to CFC-113a at ion 117 is about 1:15.

In one embodiment, the plurality of first responses for the at least one of the plurality of the first chemical fragment ions per mole of the first chemical component injected into the chromatographic column and the plurality of the second responses for the at least one of the plurality of the second chemical fragment ions per mole of the second chemical component injected into the chromatographic column comprises a first response factor corresponding to the at least one of the plurality of the first chemical fragment ions per mole of the first chemical component, a second response factor corresponding to the at least one of the plurality of the first chemical fragment ions per mole of the second chemical component, a third response factor corresponding to the at least one of the plurality of the second chemical fragment ions per mole of the first chemical component, and a fourth response factor corresponding to the at least one of the plurality of the second chemical fragment ions per mole of the second chemical component.

Another embodiment of the present invention relates to a method for quantifying overlapping measurement signals from analysis of a chemical sample, the method including receiving at a processor a plurality of spectra for a first chemical component and a second chemical component of the chemical sample, wherein the first and the second chemical components coelute from a chromatographic column; receiving at the processor a plurality of reference mass spectra for the first chemical component and the second chemical component; determining from the plurality of the reference mass spectra for the first chemical component and the second chemical component a plurality of first mass spectra signals for a plurality of first chemical fragment ions and a plurality of second mass spectra signals for a plurality of second chemical fragment ions; determining whether the plurality of the first mass spectra signals for the plurality of the first chemical fragment ions are different from the plurality of the second mass spectra signals for the plurality of the second chemical fragment ions; determining from the plurality of the first mass spectra signals for the plurality of the first chemical fragment ions a first relative abundance and from the plurality of the second mass spectra signals for the plurality of the second chemical fragment ions a second relative abundance; selecting from the plurality of the reference first mass spectra for the first chemical component and the plurality of the reference second mass spectra for the second chemical component at least one of the plurality of the first mass spectra signals for the at least one of the plurality of the first chemical fragment ions and at least one of the plurality of the second mass spectra signals for the at least one of the plurality of the second chemical fragment ions when the plurality of the first mass spectra signals for the plurality of the first chemical fragment ions are not different from the plurality of the second mass spectra signals for the plurality of the second chemical fragment ions and when the first relative abundance of the plurality of the first chemical fragment ions and the second relative abundance of the plurality of the second chemical fragment ions are not sufficient for the quantification of the overlapping measurement signals at a predetermined precision, wherein a first molar response for the first chemical component relative to the second chemical component at the at least one of the plurality of the first chemical fragment ions is different from a second molar response for the second chemical component relative to the first chemical component at the at least one of the plurality of the second chemical fragment ions; determining a plurality of first responses for the at least one of the plurality of the first chemical fragment ions per mole of the first chemical component injected into the chromatographic column and a plurality of second responses for the at least one of the plurality of the second chemical fragment ions per mole of the second chemical component injected into the chromatographic column; and determining a first measurement signal peak response corresponding to the plurality of the first responses for the at least one of the plurality of the first chemical fragment ions per mole of the first chemical component and a second measurement signal peak response corresponding to the plurality of the second responses for the at least one of the plurality of the first chemical fragment ions per mole of the first chemical component. More particularly, the first chemical component is CFC-113 and the second chemical component is CFC-113a.

In one embodiment, the first chemical component is CFC-113 and the second chemical component is CFC-113a, wherein the first molar response for CFC-113 relative to CFC-113a at ion 103 is about 5:1, and wherein the second molar response of CFC-113 relative to CFC-113a at ion 117 is about 1:15.

In some embodiments, the plurality of first responses for the at least one of the plurality of the first chemical fragment ions per mole of the first chemical component and the plurality of the second responses for the second chemical fragment ions per mole of the second chemical component comprises a first response factor corresponding to the at least one of the plurality of the first chemical fragment ions per mole of the first chemical component, a second response factor corresponding to the at least one of the plurality of the first chemical fragment ions per mole of the second chemical component, a third response factor corresponding to the at least one of the plurality of the second chemical fragment ions per mole of the first chemical component, and a fourth response factor corresponding to the at least one of the plurality of the second chemical fragment ions per mole of the second chemical component.

Embodiments of the present invention also relate to a method for quantifying overlapping measurement signals from analysis of a chemical sample, the method including receiving at a processor a plurality of spectra for a first chlorofluorocarbon component and a second chlorofluorocarbon component, wherein the first and the second chlorofluorocarbon components coclute from a chromatographic column, wherein each of the plurality of the spectra comprises a scan number, response and mass-to-charge ratio of charged particles; receiving at the processor a plurality of reference mass spectra for the first chlorofluorocarbon component and the second chlorofluorocarbon component; determining from the plurality of the reference mass spectra for the first chlorofluorocarbon component and the second chlorofluorocarbon component a plurality of first mass spectra signals for a plurality of first chlorofluorocarbon fragment ions and a plurality of second mass spectra signals for a plurality of second chlorofluorocarbon fragment ions; determining whether the plurality of the first mass spectra signals for the plurality of the first chlorofluorocarbon fragment ions are different from the plurality of the second mass spectra signals for the plurality of the second chlorofluorocarbon fragment ions; determining from the plurality of the first mass spectra signals for the plurality of the first chlorofluorocarbon fragment ions a first relative abundance and from the plurality of the second mass spectra signals for the plurality of the second chlorofluorocarbon fragment ions a second relative abundance; selecting from the plurality of the reference first mass spectra for the first chlorofluorocarbon component and the plurality of the reference second mass spectra for the second chlorofluorocarbon component at least one of the plurality of the first mass spectra signals for the at least one of the plurality of the first chlorofluorocarbon fragment ions and at least one of the plurality of the second mass spectra signals for the at least one of the plurality of the second chlorofluorocarbon fragment ions when the plurality of the first mass spectra signals for the plurality of the first chlorofluorocarbon fragment ions are not different from the plurality of the second mass spectra signals for the plurality of the second chlorofluorocarbon fragment ions and when the first relative abundance of the plurality of the first chlorofluorocarbon fragment ions and the second relative abundance of the plurality of the second chlorofluorocarbon fragment ions are not sufficient for the quantification of the overlapping measurement signals at a predetermined precision, wherein a first molar response for the first chlorofluorocarbon component relative to the second chlorofluorocarbon component at the at least one of the first chlorofluorocarbon fragment ions is different from a second molar response for the second chlorofluorocarbon component relative to the first chlorofluorocarbon component at the at least one of the plurality of the second chemical fragment ions; determining a plurality of first responses for the at least one of the plurality of the first chlorofluorocarbon fragment ions per mole of the first chlorofluorocarbon component and a plurality of second responses for the at least one of the plurality of the second chlorofluorocarbon fragment ions per mole of the second chlorofluorocarbon component; and determining a first measurement signal peak response corresponding to the plurality of the first responses for the at least one of the plurality of the first chlorofluorocarbon fragment ions per mole of the first chlorofluorocarbon component and a second measurement signal peak response corresponding to the plurality of the second responses for the at least one of the plurality of the first chlorofluorocarbon fragment ions per mole of the first chlorofluorocarbon component.

### BRIEF DESCRIPTION OF DRAWINGS

FIG. 1 illustrates a schematic representation of an instrumental analysis system used for separating the components of a multi-component sample mixture and then detecting the components in a multi-channel mode.

FIG. 2 illustrates a flow chart showing a method in accordance with an embodiment of the present invention for determining mole fractions of individual chemical components in a sample when both normal separation conditions are not achieved.

FIG. 3 illustrates a plot comparing mole fractions of CFC-113a determined using a method in accordance with an embodiment of the present invention (unfilled diamonds) with mole fractions of CFC-113a determined using a prior art method.

FIG. 4 illustrates an exemplary application of the method in accordance with embodiments of the present invention for overlapping chemicals signal quantification for the analysis of over 1000 air samples collected at sites across the globe since late 2023.

FIG. 5(A) illustrates sample ion chromatograms obtained for ambient air showing ion responses for three different fragment ions (top panel), FIG. 5(B) illustrates responses of those same ions after normalization to rescale the results so that all ion peaks are the same height (middle panel), and FIG. 5(C) illustratres an overlay of ion chromatograms from two separate analyses in which standards containing only CFC-113a (labeled A with ion traces indicated by dashed lines) and only CFC-113 (labeled B with ion traces indicated by solid lines) were injected (bottom panel).

FIG. 6 illustrates the precision achieved in quantifying the mole fraction of CFC-113a in routine analyses of air samples collected at a remote site using overlapping chemical signals quantification method in accordance with embodiments of the present invention.

FIG. 7 illustrates exemplary results for atmospheric CFC-113 mole fraction with use of conventional methods in comparison with the bias-free results for CFC-113 obtained by using overlapping chemical signals quantification methods in accordance with embodiments of the present invention.

FIG. 8 illustrates exemplary results for the biases added to global emission rates of CFC-113 derived from the atmospheric mole fractions of CFC-113 shown in FIG. 3 when measured using the single ions 103 or 153, relative to using overlapping chemical signals quantification method in accordance with embodiments of the present invention (where bias=zero for all years).

### DESCRIPTION OF INVENTION

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention, and do not delimit the scope of the present invention. Reference will now be made to the drawings wherein like numerals refer to like elements throughout. As used herein, expressing concentration in terms of mole fraction means representing the amount of a substance in a sample mixture as the ratio of its moles to the total moles of all substances present in that sample.

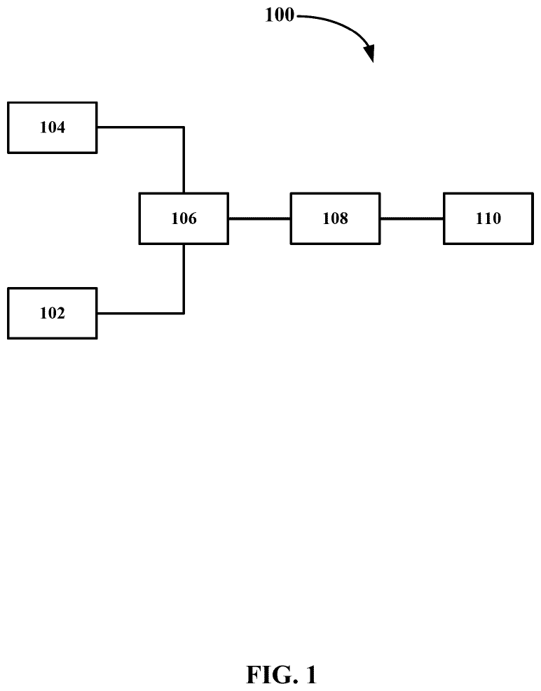

Referring now to the drawings, and more particularly, to FIG. 1, there is shown a system for analyzing environmental samples that involves detecting and independently quantifying overlapping signals from chemical fragments in the environment samples, generally designated 100 and schematically showing an embodiment of the present invention. Overlapping chemical signals detection system 100 includes a sample introduction mechanism 102, a carrier fluid source 104, a chromatograph 106, a detector 108, and a processor 110 for recording instrument responses to samples being analyzed.

Chromatograph 106 receives environmental samples via sample introduction mechanism 102 as discrete samples. In one embodiment of the present invention, chromatograph 106 is a gas chromatograph. In another embodiment of the present invention, chromatograph 106 is a liquid chromatograph. In yet another embodiment of the present invention, chromatograph 106 is a supercritical fluid chromatograph. Samples from sample introduction port 102 may be combined with a carrier fluid from carrier fluid source 104. Carrier fluid from carrier fluid source 104 can be gas, liquid, supercritical fluid, and the like. In embodiments wherein chromatograph 106 is a gas chromatograph, the carrier gas from carrier gas source 104 is used to transport samples to and within chromatograph 106. In some embodiments of the present invention wherein chromatograph 106 a gas chromatograph, about 1.7 ml/min of helium is used as a carrier gas, with a temperature ramp of the chromatography column from about −55 deg. C to about 200 deg. C with each injection, for optimal performance. Flow is maintained despite viscosity changes in the carrier fluid by programming the head pressure on the column. The flow rate may be determined by the length and diameter of the capillary column used for separation, the carrier fluid type and operating temperature. Compounds may interact with a specific film on the inside of the capillary column, and may be separated by volatility and polarity as they pass through.

Chromatograph 106 may include an injector and a column. The injector may serve as an inlet and be configured to allow for the injection of the sample and carrier fluid suitable for chromatography and separation to occur on the column. The column may include an open tube capillary column that may be used to enhance resolution, reduce column bleed and reduce analysis time. In an exemplary embodiment of the present invention, capillary column is 60 meters long having 0.25 mm ID, with 1 micron film made from 5% phenyl-methyl silicone. Low column bleed may reduce the background signal for measurements of components at low concentrations and higher resolution increases the time between separated compounds.

Operating parameters of chromatograph 106, such as carrier fluid composition, temperature, flow rate, pressure, may be optimized to enable separation of as many of the components of a sample as possible as they exit the column. Operating parameters of chromatograph 106 may be configured on chromatograph 106 or on processor 110 to maximize peak resolution and separation. Co-elution of peaks may not be desirable as this may cause biases (or inaccuracy) in the quantification of the concentrations (or mole fractions) of chemicals measured in the sample. Chromatograph 106 may be manipulated to provide different separation characteristics that completely resolve samples into all their discrete components, although this is highly unlikely for complex environmental samples or in samples where separation is sought for components with very similar physical and chemical characteristics, for example, structural isomers.

Detector 108 may be used to monitor the effluent from the column in chromatograph 106 and to determine the abundance of individual compounds in the sample. Method in accordance with embodiments of the present invention for quantifying overlapping chemical signals may be used when detector 108 is used after separation in the chromatograph. Detector 108 is capable of monitoring with a range of efficiencies multiple signals produced by the overlapping chemicals. In one embodiment of the present invention, detector 108 for measuring response on different channels or ions may be a mass spectrometer. Exemplary mass spectrometers that can be used in embodiments in accordance with the present invention include an accelerator mass spectrometer, isotopic ratio mass spectrometer, ion-trap mass spectrometer, time-of-flight mass spectrometer, and the like. Detector 108 may be directly coupled to the end of chromatograph 106 column such that the carrier fluid stream exiting chromatograph 106 is passed to detector 108. In embodiments wherein detector 108 is a mass spectrometer, ion fragments formed from chemicals after bombardment by a 70 e V electron beam in the source region can be independently monitored. Ions are formed and separated and detected in the “multi-channel” mass spectrometer detector. Different chemicals typically produce different ion fragments having different masses as they decompose after being bombarded with the high-energy electrons. In those instances where unique ions are generated by chemicals that have not been separated by the chromatography, accurate and un-biased quantitation may be possible through monitoring those unique ions. Some chemicals, particularly those that are structural isomers, often produce identical ions in this fragmentation process and accurate, unbiased quantitation becomes possible with the use of the method in accordance with embodiments of the present invention for the quantification of chemical signals. The data obtained by detector 108 from monitoring the effluent from the column in chromatograph 106 may be recorded by processor 110, and may include a scan number, response and mass-to-charge ratio of charged particles.

Processor 110 may be a personal computer, or any suitable stand-alone system or a system connected to a network, for recording and managing of the data produced by detector 108. Processor 110 may include, among other things, a user input device, memory, display, processor, and network interface. A user input device may be configured to allow users to control processor 110. Memory, such as a hard disk, removable disk, RAM, flash memory, may be used to store data produced by detector 108, or produced by chromatograph 106. A display such as an LCD screen or any other suitable display may be used by the operator of the system to view information about processor 110 or data recorded by detector 108. Processor 110 may be used to control various devices or perform any necessary computations. A network interface may be used to send the data recorded by detector 108 or any other information to other systems connected to a network.

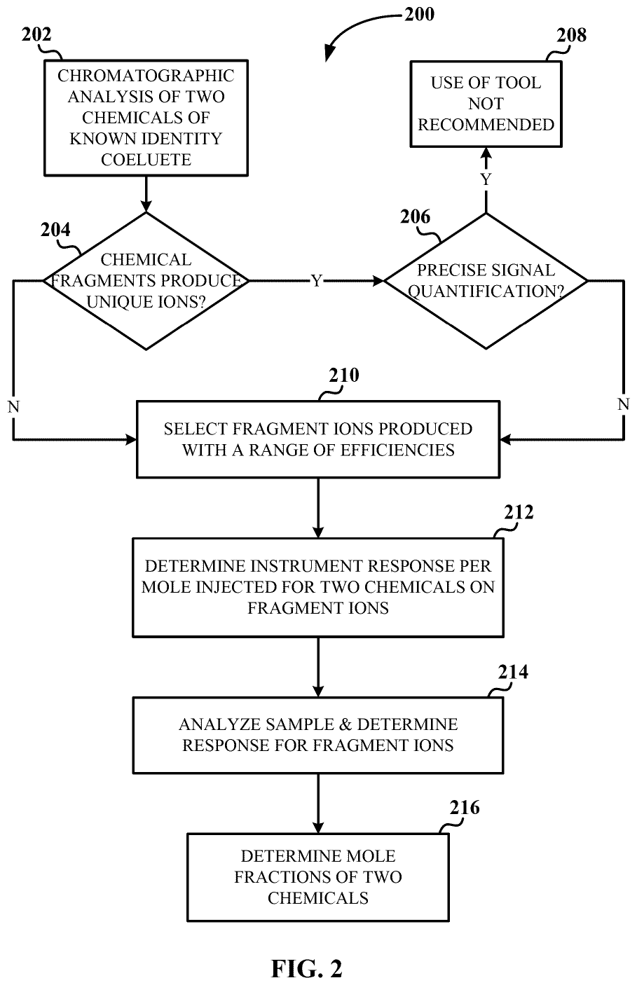

FIG. 2 is a flowchart illustrating a method 200 for quantifying overlapping chemical signals resulting in the environmental analysis of complex samples using chromatography (gas, liquid, supercritical fluid, and the like) coupled to a multi-channel detector such as mass spectrometry, wherein overlapping chemical signals quantification method 200 determines the mole fractions of chemical components in a sample when both normal separation conditions are not achieved. Overlapping chemical signals quantification method 200 receives, at step 202, data related to chromatographic analysis in which two chemicals, A and B, of known identity coclute. The data obtained at step 202 includes chromatographic analysis of chemicals A and B using chromatograph 106 followed by detection of fragment ions X and Y produced by chemicals A and B by detector 108, and recording signal response data by processor 110.

At step 204, overlapping chemical signals quantification method 200 inspects reference mass spectra for each chemical compound (either from established mass spectral libraries or from overlapping chemical signals detection system 100 in separate analyses of cach co-cluting chemical in synthetic mixtures containing single component performed on the instrumentation) and determines whether the chemical fragments to produce unique ions that allow for unbiased quantification despite lack of separation in time. If overlapping chemical signals quantification method 200 determines, at step 204, that the chemicals fragments produce unique ions that allow for separate quantification despite lack of separation in time, then, at step 206, overlapping chemical signals quantification method 200 determines whether the unique ions are produced with sufficient relative abundance to allow for quantification at the desired precision. If it is determined at step 206 that the signals generated have sufficient relative abundance for quantification at the desired precision, then, at step 208, it is recommended that no further processing using overlapping chemical signals quantification method 200 is required.

If it is determined at step 204 that the chemicals A and B do not produce unique ions that allow for unbiased and separate quantification given the lack of separation in time, or it is determined at step 206 that the signals from unique ions are not sufficiently abundant for quantification at the desired precision, then, at step 210, overlapping chemical signals quantification method 200 further inspects the reference mass spectra for chemical fragmentation patterns for A and B, and selects two or more fragment ions (X and Y) that are produced by chemicals A and B. In a preferred embodiment of the present invention, overlapping chemical signals quantification method 200 selects, at step 210, ion X that is produced relatively efficiently, or in relative abundance, by chemical A but not by chemical B, and selects ion Y that is produced relatively efficiently, or in relative abundance, by chemical B but not by chemical A. At step 210, overlapping chemical signals quantification method 200 selects two ions such that molar response at one ion is largest for one of the two compounds, and the molar response at the second ion is largest for the other compound. Although larger discrimination in molar responses for the chemicals at these ions is preferred for better quantification of overlapping chemical signals, quantification of overlapping chemical signals quantification can be also achieved using a method in accordance with embodiments of the present invention even if the relative response at one ion is similar, as long as the response at the other ion is quite different. In an exemplary embodiment, the ratio of molar response at ion 153 for CFC-113 to the molar response at ion 153 for CFC-113a was about 0.9:1, and when combined with the relative response at ion 117 (1:15), the results were still robust. In another exemplary embodiment, the molar response for CFC-113 relative to CFC-113a at ion 103 was about 5:1, whereas the molar response of CFC-113 relative to CFC-113a at ion 117 was about 1:15.

At step 212, overlapping chemical signals quantification method 200 determines instrument response (peak area) for ions X and Y per mole of injected chemicals A and B. At step 212, overlapping chemical signals quantification method 200 determines molar response factors from analyses of two standards, one with compound A and not B, and the other containing compound B and not A. The separate analysis allow for the determination of four response factors, RF1, RF2, RF3 and RF4, at step 212, wherein RF1 is the response (peak area) at ion X per mole of chemical A injected on column; RF2 is the response (peak area) at ion X per mole of chemical B injected on column; RF3 is the response (peak area) at ion Y per mole of chemical A injected on column; and RF4 is the response (peak area) at ion Y per mole of chemical B injected on column.

At step 214, overlapping chemical signals quantification method 200 determines the peak responses R_{X} and R_{Y} (peak areas) measured at ions X and Y from the summed contributions from chemicals A and B. Given that the response R from a compound is the product of its mole fraction and its molar response (as response/mole), and R_{X} and R_{Y} represent the sum of responses from both chemicals at the different ions, the ion responses can be expressed as follows:

$${R}_{X} = {MF}_{A} \times RF 1 + {MF}_{B} \times RF 2$$

$${R}_{Y} = {MF}_{A} \times RF 3 + {MF}_{B} \times RF 4$$

The mole fractions of chemical A (MF_{A}) and the mole fractions of chemical B (MF_{B}) in the sample are determined using the following equations:

$${MF}_{A} = [ {R}_{X} - {R}_{Y} \times ( RF 2 / RF 4 ) ] / [ RF 1 - RF 3 \times ( RF 2 / RF 4 ) ]$$

$${MF}_{B} = ( {R}_{Y} - {MF}_{A} \times RF 3 ) / RF 4$$

Reference to the specific examples which follow and included herein are intended to provide a clearer understanding of methods in accordance with embodiments of the present invention and its capabilities. The examples should not be construed as a limitation upon the scope of the present invention.

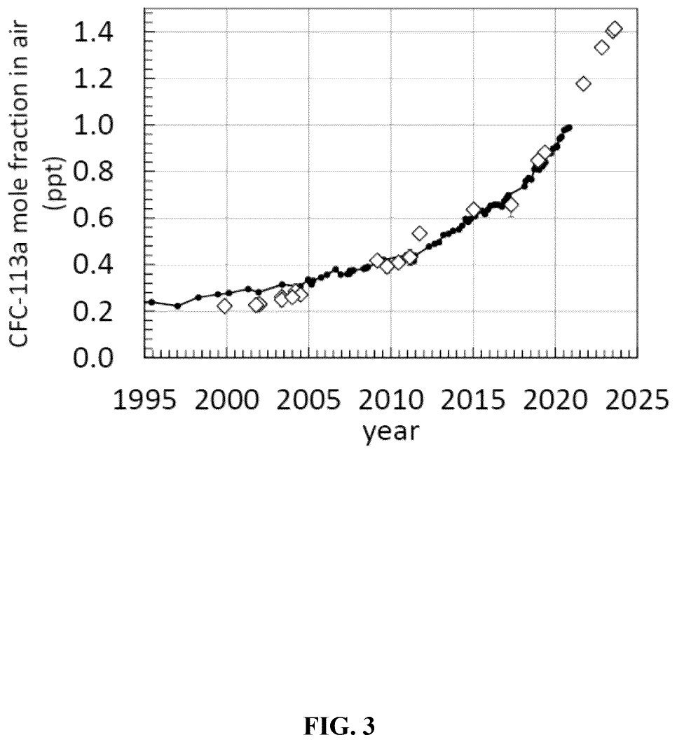

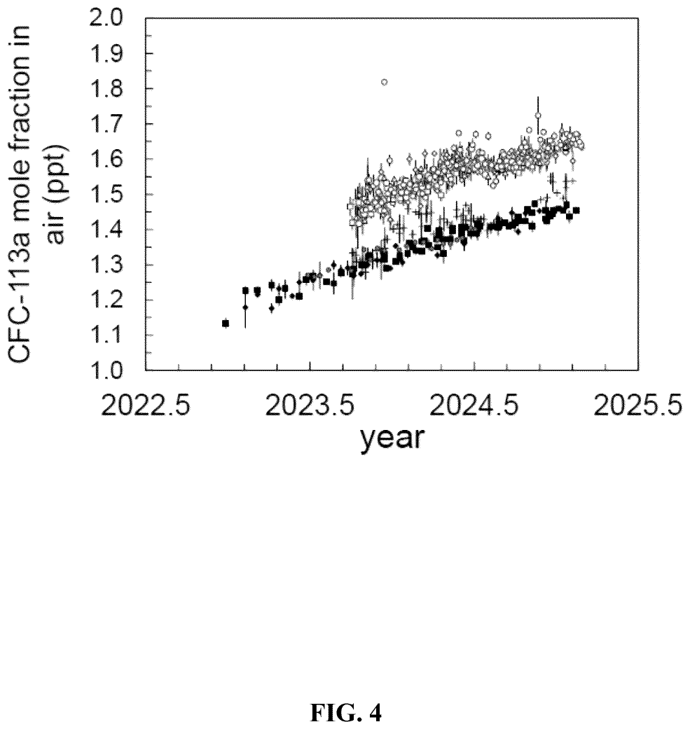

The following example illustrates quantifying overlapping chemical signals of difficult to separate isomers of chlorinated fluorocarbons (CFC), CCl₂FCClF₂(CFC-113) and CCl₃CF₃ (CFC-113a), in environmental air analysis without any substantial modification of chromatographic analysis parameters and using a method in accordance with embodiments of the present invention. Atmospheric measurements of CFC-113 in today's atmosphere will likely be biased by coelution of its rapidly increasing isomer CFC-113a because commonly used chromatographic columns and column conditions do not allow for separation in time, and the abundant fragment ions produced from CFC-113 in the source of a mass spectrometer are also produced by CFC-113a. The measurement bias on CFC-113 caused by the underlying response from the isomer CFC-113a is significant and this bias is also ion-and instrument-dependent. Furthermore, CFC-113a has only periodically been measured in ambient air samples because its signal is swamped by CFC-113 at all ions, given that the ambient air mole fraction of CFC-113 is currently about 65 ppt and CFC-113a is approximately 1.5 ppt. FIGS. 3 and 4 illustrate CFC-113a mole fractions obtained by tracking response at 2 ions and tracking four different response factors over time using a chemical signals quantification method in accordance with embodiments of the present invention. FIG. 3 illustrates a plot of historical changes in the atmospheric mole fractions measured for CFC-113a in parts per trillion (ppt), the less abundant isomer in the CFC-113 /CFC-113a pair. One set of results shown in FIG. 3 (solid circles and line) were obtained by a configuration of the GCMS parameters to allow for separation in time, and are deemed reference data when “normal separation conditions” were met. The results obtained using overlapping chemical signals quantification method in accordance with embodiments of the present invention with minimal instrument disruption are shown as unfilled diamonds in FIG. 3, and are found to be consistent with this earlier work. The results obtained demonstrate that the method for chemicals signal quantification provides for bias-free atmospheric measurements. FIG. 4 illustrates the successful routine application of overlapping chemicals signal quantification method in accordance with embodiments of the present invention for the analysis of over 1000 air samples collected at sites across the globe since late 2023. Overlapping chemicals signal quantification method 200 enables regular measurements with minimal disruptions to normal instrument operating procedures and standard instrumental conditions. The results shown in FIG. 4 provide new insights into the global mole-fraction distribution and trends of this internationally regulated chemical.

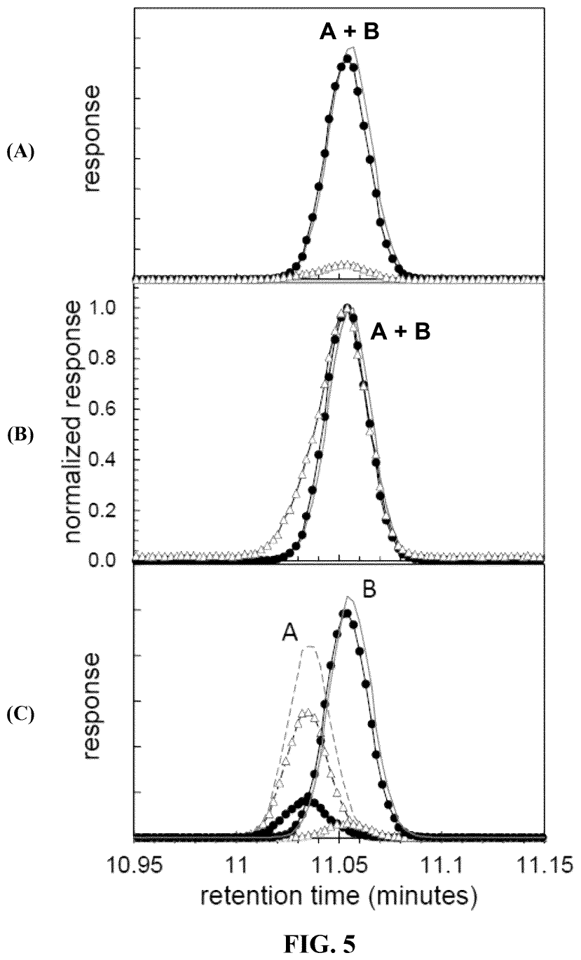

Overlapping chemical signals quantification method in accordance with embodiments of the present invention was used to deconvolve the CFC-113 and CFC-113a isomers in atmospheric samples collected since late 2023 by measuring peak areas at ions 103 and 117, given the large relative response differences they have for the different chemicals, and by regularly measuring the four molar response factors. FIG. 5 illustrates ion chromatograms obtained by a mass spectrometer at ions 103, 117 and 153, and shows influence from both CFC-113 and CFC-113a in ambient air and from single component standards. FIG. 5(A) illustrates sample ion chromatograms obtained for ambient air showing ion responses for three different fragment ions, FIG. 5(B) illustrates responses of those same ions after normalization to rescale the ion chromatograms so that all ion peaks are the same height, and FIG. 5(C) illustratres an overlay of ion chromatograms from two separate analyses in which standards containing only CFC-113a at 29 ppt (labeled A with ion traces indicated by dashed lines) and only CFC-113 at 69 ppt (labeled B with ion traces indicated by solid lines) were injected. The signals for 3 different chemicals fragment ions appear in every panel: ion 103 as filled circles connected by lines; ion 117 as the white-filled triangles; and ion 153 as the gray line having no symbols.

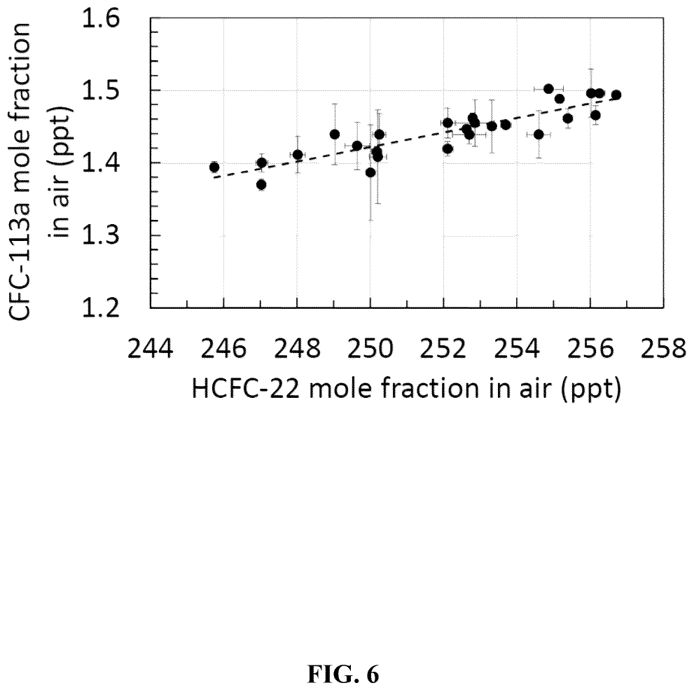

FIG. 6 illustrates the precision achieved in quantifying the mole fraction of the lesser-abundant isomer, CFC-113a, in routine analyses of air samples collected at a remote site using overlapping chemical signals quantification method in accordance with embodiments of the present invention. The high precision (1 standard deviation of replicate injections˜0.02 ppt) afforded by the overlapping chemical signals quantification method enables heretofore unseen correlations (r²=0.77 in these data) to be measured with other halogenated hydrocarbons, in this case with HCFC-22 (CHCl₂), that have implications for identifying upwind regions that are contributing to the rapid increase measured in the atmosphere for CFC-113a. In the absence of use of the overlapping chemical signals quantification method, these increases could have been erroneously attributed to CFC-113. The error bars in FIG. 6 represent the precision of the analysis, which is 0.02 ppt on average for CFC-113a in these results.

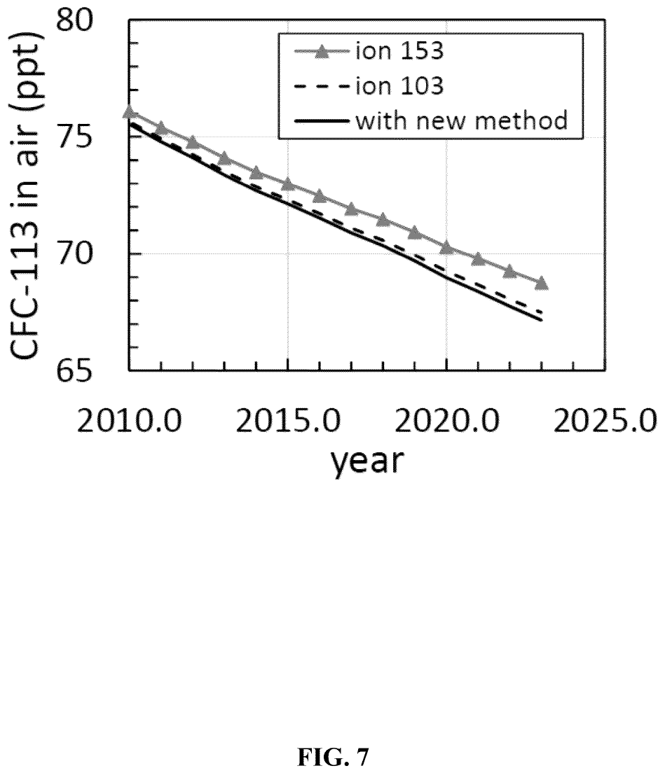

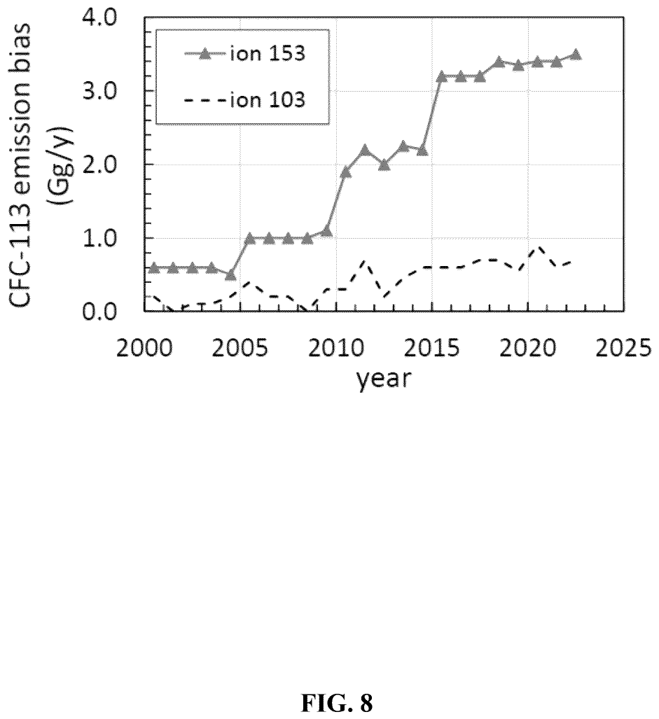

In estimating the bias from co-clution of CFC-113a on the historical mole fractions and emissions of CFC-113, it is assumed that response factors determined are relevant for all years and the added response at CFC-113a is calculated from the true CFC-113a history. Use of ion 103 to measure CFC-113 using conventional methods leads to a small bias over time and use of ion 153 suggests a higher bias, particularly in recent years, as shown in FIGS. 7 and 8. Further, it has been determined that electron capture detectors, commonly used in the chromatographic analysis of CFCs in air, are even more sensitive to CFC-113a compared to CFC-113 and would lead to even larger biases in nominal measurements and derived global emissions for CFC-113 if these isomers were not separated chromatographically. FIG. 7 illustrates exemplary results for historical atmospheric CFC-113 mole fractions with use of conventional methods that suffer from bias associated with co-elution of CFC-113a when monitoring ion fragment at m/z=153 or 103. Also shown are the bias-free results for CFC-113 obtained by using overlapping chemical signals quantification methods in accordance with embodiments of the present invention. FIG. 8 illustrates exemplary results for the bias added to global emission rates of CFC-113 derived from the atmospheric mole fractions of CFC-113 shown in FIG. 7. FIG. 8 shows the bias in emission derived using the two different ions vs what is derived from using methods in accordance with embodiments of the present invention.

A method in accordance with embodiments of the present invention to independently quantify mole fractions (concentrations or abundances) of CFC-113 and CFC-113a in ambient air can be applied to provide independent quantification of other coeluting gases or isomers thereof, such as CFC-114 and CFC-114a. Use of the method in accordance with embodiments of the present invention in measuring CFC-113a allows for precise and accurate measurements and, therefore, the quantification of small atmospheric mole fraction gradients and trends over time. In one embodiment, use of the method in accordance with embodiments of the present invention in measuring CFC-113a allows for the quantification of about 0.02 ppt atmospheric differences and gradients. Exemplary results show that the method in accordance with embodiments of the present invention can be used to measure global distribution that suggests source regions and confirm rapid increases over time.

A method in accordance with embodiments of the present invention have been used in estimating interferences in historical records of CFC-113 atmospheric abundance changes (as mole fraction) and global emissions estimated from those abundance records. The estimated results show a bias in global CFC-113 emissions of up to 3.5 Gg/yr (FIG. 8), which represents an error of approximately 100% in global CFC-113 emissions.

Overlapping chemical signals quantification method in accordance with embodiments of the present invention allows a simple approach for independently measuring chemicals that are unresolved in conventional analysis using chromatography with mass spectrometric detection (by time and mass). In the absence of the present invention, at least one of these conditions has typically been required for being able to independently quantify the amount of cach component without interference by the other. Overlapping chemical signals quantification method described herein allows one to achieve this separation without the need for extensive modification of physical analysis conditions (analysis time, chromatography column), or the purchase of more sophisticated (expensive) instrumentation. The approach could be added to software packages for quantifying chromatographic signals and that are currently used extensively in the environmental analysis field. A method in accordance with embodiments of the present invention has utility for assessing compliance with international agreements related to stratospheric ozone depletion.

Overlapping chemical signals quantification methods in accordance with one or more embodiments of the present invention can be adapted to a variety of configurations. It is thought that overlapping chemical signals quantification methods in accordance with various embodiments of the present invention and many of its attendant advantages will be understood from the foregoing description and it will be apparent that various changes may be made without departing from the spirit and scope of the invention or sacrificing all its material advantages, the form hereinbefore described being merely a preferred or exemplary embodiment thereof.

Those familiar with the art will understand that embodiments of the invention may be employed, for various specific purposes, without departing from the essential substance thereof. The description of any one embodiment given above is intended to illustrate an example rather than to limit the invention. This above description is not intended to indicate that any one embodiment is necessarily preferred over any other one for all purposes, or to limit the scope of the invention by describing any such embodiment, which invention scope is intended to be determined by the claims, properly construed, including all subject matter encompassed by the doctrine of equivalents as properly applied to the claims.

What is claimed is:

1. A method for quantifying overlapping measurement signals from analysis of a chemical sample, the method comprising:
receiving at a processor a plurality of spectra for a first chemical component and a second chemical component of the chemical sample, wherein the first and the second chemical components coelute from a chromatographic column;
receiving at the processor a plurality of reference spectra for the first chemical component and the second chemical component;
determining from the plurality of the reference spectra for the first chemical component and the second chemical component a plurality of first fragment ion signals for a plurality of first chemical fragment ions and a plurality of second fragment ion signals for a plurality of second chemical fragment ions;
determining whether the plurality of the first fragment ion signals for the plurality of the first chemical fragment ions are different from the plurality of the second fragment ion signals for the plurality of the second chemical fragment ion;
selecting from the plurality of the reference spectra for the first chemical component and the second chemical component at least one of the plurality of the first fragment ion signals for at least one of the plurality of the first chemical fragment ions and at least one of the plurality of the second fragment ion signals for the at least one of the plurality of the second chemical fragment ions when the plurality of the first fragment ion signals for the plurality of the first chemical fragment ions are not different from the plurality of the second fragment ion signals for the plurality of the second chemical fragment ions, wherein a first molar response for the first chemical component relative to the second chemical component at the at least one of the plurality of the first chemical fragment ions is different from a second molar response for the first chemical component relative to the second chemical component at the at least one of the plurality of the second chemical fragment ions;
determining a plurality of first responses for the at least one of the plurality of the first chemical fragment ions per mole of the first chemical component injected into the chromatographic column and a plurality of second responses for the at least one of the plurality of the second chemical fragment ions per mole of the second chemical component injected into the chromatographic column; and
determining a first measurement signal peak response corresponding to the plurality of the first responses for the at least one of the plurality of the first chemical fragment ions per mole of the first chemical component and a second measurement signal peak response corresponding to the plurality of the second responses for the at least one of the plurality of the first chemical fragment ions per mole of the first chemical component.

2. The method of claim 1, wherein the first chemical component is CFC-113 and the second chemical component is CFC-113a.

3. The method of claim 1, further comprising determining a first relative abundance of the plurality of the first fragment ions from the plurality of the first fragment ion signals and a second relative abundance of the plurality of the second fragment ions from plurality of the second fragment ion signals.

4. The method of claim 3, wherein the selecting from the plurality of the reference spectra for the first chemical component and the second chemical component at least one of the plurality of the first fragment ion signals for the at least one of the plurality of the first chemical fragment ions and at least one of the plurality of the second fragment ion signals for the at least one of the plurality of the second chemical fragment ions when the first relative abundance of the plurality of the first chemical fragment ions and the second relative abundance of the plurality of the second chemical fragment ions are not sufficient for the quantification of the overlapping measurement signals at a predetermined precision.

5. The method of claim 1, wherein the selecting the at least one of the plurality of the first fragment ion signals and the at least one of the plurality of the second fragment ion signals comprises determining the first chemical fragment ion created by the first chemical component in relative abundance to the second chemical component and the second chemical fragment ion created by the second chemical component in relative abundance to the first chemical component.

6. The method of claim 1, wherein the first molar response for the first chemical component relative to the second chemical component at the at least one of the plurality of the first chemical fragment ions is greater than the second molar response for the first chemical component relative to the second chemical component at the at least one of the plurality of the second chemical fragment ions.

7. The method of claim 1, wherein the first chemical component is CFC-113 and the second chemical component is CFC-113a, wherein the first molar response for CFC-113 relative to CFC-113a at ion 103 is about 5:1, and wherein the second molar response of CFC-113 relative to CFC-113a at ion 117 is about 1:15.

8. The method of claim 1, wherein the plurality of first responses for the at least one of the plurality of the first chemical fragment ions per mole of the first chemical component injected into the chromatographic column and the plurality of the second responses for the at least one of the plurality of the second chemical fragment ions per mole of the second chemical component injected into the chromatographic column comprises a first response factor corresponding to the at least one of the plurality of the first chemical fragment ions per mole of the first chemical component, a second response factor corresponding to the at least one of the plurality of the first chemical fragment ions per mole of the second chemical component, a third response factor corresponding to the at least one of the plurality of the second chemical fragment ions per mole of the first chemical component, and a fourth response factor corresponding to the at least one of the plurality of the second chemical fragment ions per mole of the second chemical component.

9. The method of claim 1, wherein each of the plurality of the spectra comprises a scan number, response and mass-to-charge ratio of charged particles.

10. A method for quantifying overlapping measurement signals from analysis of a chemical sample, the method comprising:
receiving at a processor a plurality of spectra for a first chemical component and a second chemical component of the chemical sample, wherein the first and the second chemical components coelute from a chromatographic column;
receiving at the processor a plurality of reference mass spectra for the first chemical component and the second chemical component;
determining from the plurality of the reference mass spectra for the first chemical component and the second chemical component a plurality of first mass spectra signals for a plurality of first chemical fragment ions and a plurality of second mass spectra signals for a plurality of second chemical fragment ions;
determining whether the plurality of the first mass spectra signals for the plurality of the first chemical fragment ions are different from the plurality of the second mass spectra signals for the plurality of the second chemical fragment ions;
determining from the plurality of the first mass spectra signals for the plurality of the first chemical fragment ions a first relative abundance and from the plurality of the second mass spectra signals for the plurality of the second chemical fragment ions a second relative abundance;
selecting from the plurality of the reference first mass spectra for the first chemical component and the plurality of the reference second mass spectra for the second chemical component at least one of the plurality of the first mass spectra signals for the at least one of the plurality of the first chemical fragment ions and at least one of the plurality of the second mass spectra signals for the at least one of the plurality of the second chemical fragment ions when the plurality of the first mass spectra signals for the plurality of the first chemical fragment ions are not different from the plurality of the second mass spectra signals for the plurality of the second chemical fragment ions and when the first relative abundance of the plurality of the first chemical fragment ions and the second relative abundance of the plurality of the second chemical fragment ions are not sufficient for the quantification of the overlapping measurement signals at a predetermined precision, wherein a first molar response for the first chemical component relative to the second chemical component at the at least one of the plurality of the first chemical fragment ions is different from a second molar response for the second chemical component relative to the first chemical component at the at least one of the plurality of the second chemical fragment ions;
determining a plurality of first responses for the at least one of the plurality of the first chemical fragment ions per mole of the first chemical component injected into the chromatographic column and a plurality of second responses for the at least one of the plurality of the second chemical fragment ions per mole of the second chemical component injected into the chromatographic column; and
determining a first measurement signal peak response corresponding to the plurality of the first responses for the at least one of the plurality of the first chemical fragment ions per mole of the first chemical component and a second measurement signal peak response corresponding to the plurality of the second responses for the at least one of the plurality of the first chemical fragment ions per mole of the first chemical component.

11. The method of claim 10, wherein the first chemical component is CFC-113 and the second chemical component is CFC-113a.

12. The method of claim 10, wherein the selecting the at least one of the plurality of the first mass spectra signals and the at least one of the plurality of the second mass spectra signals comprises determining the first chemical fragment ion created by the first chemical component in relative abundance to the second chemical component and the second chemical fragment ion created by the second chemical component in relative abundance to the first chemical component.

13. The method of claim 10, wherein the first molar response for the first chemical component relative to the second chemical component at the at least one of the plurality of the first chemical fragment ions is greater than the second molar response for the first chemical component relative to the second chemical component at the at least one of the plurality of the second chemical fragment ions.

14. The method of claim 10, wherein the first chemical component is CFC-113 and the second chemical component is CFC-113a, wherein the first molar response for CFC-113 relative to CFC-113a at ion 103 is about 5:1, and wherein the second molar response of CFC-113 relative to CFC-113a at ion 117 is about 1:15.

15. The method of claim 10, wherein the plurality of first responses for the at least one of the plurality of the first chemical fragment ions per mole of the first chemical component and the plurality of the second responses for the second chemical fragment ions per mole of the second chemical component comprises a first response factor corresponding to the at least one of the plurality of the first chemical fragment ions per mole of the first chemical component, a second response factor corresponding to the at least one of the plurality of the first chemical fragment ions per mole of the second chemical component, a third response factor corresponding to the at least one of the plurality of the second chemical fragment ions per mole of the first chemical component, and a fourth response factor corresponding to the at least one of the plurality of the second chemical fragment ions per mole of the second chemical component.

16. A method for quantifying overlapping measurement signals from analysis of a chemical sample, the method comprising:
receiving at a processor a plurality of spectra for a first chlorofluorocarbon component and a second chlorofluorocarbon component, wherein the first and the second chlorofluorocarbon components coelute from a chromatographic column, wherein each of the plurality of the spectra comprises a scan number, response and mass-to-charge ratio of charged particles;
receiving at the processor a plurality of reference mass spectra for the first chlorofluorocarbon component and the second chlorofluorocarbon component;
determining from the plurality of the reference mass spectra for the first chlorofluorocarbon component and the second chlorofluorocarbon component a plurality of first mass spectra signals for a plurality of first chlorofluorocarbon fragment ions and a plurality of second mass spectra signals for a plurality of second chlorofluorocarbon fragment ions;
determining whether the plurality of the first mass spectra signals for the plurality of the first chlorofluorocarbon fragment ions are different from the plurality of the second mass spectra signals for the plurality of the second chlorofluorocarbon fragment ions;
determining from the plurality of the first mass spectra signals for the plurality of the first chlorofluorocarbon fragment ions a first relative abundance and from the plurality of the second mass spectra signals for the plurality of the second chlorofluorocarbon fragment ions a second relative abundance;
selecting from the plurality of the reference first mass spectra for the first chlorofluorocarbon component and the plurality of the reference second mass spectra for the second chlorofluorocarbon component at least one of the plurality of the first mass spectra signals for the at least one of the plurality of the first chlorofluorocarbon fragment ions and at least one of the plurality of the second mass spectra signals for the at least one of the plurality of the second chlorofluorocarbon fragment ions when the plurality of the first mass spectra signals for the plurality of the first chlorofluorocarbon fragment ions are not different from the plurality of the second mass spectra signals for the plurality of the second chlorofluorocarbon fragment ions and when the first relative abundance of the plurality of the first chlorofluorocarbon fragment ions and the second relative abundance of the plurality of the second chlorofluorocarbon fragment ions are not sufficient for the quantification of the overlapping measurement signals at a predetermined precision, wherein a first molar response for the first chlorofluorocarbon component relative to the second chlorofluorocarbon component at the at least one of the first chlorofluorocarbon fragment ions is different from a second molar response for the second chlorofluorocarbon component relative to the first chlorofluorocarbon component at the at least one of the plurality of the second chemical fragment ions;
determining a plurality of first responses for the at least one of the plurality of the first chlorofluorocarbon fragment ions per mole of the first chlorofluorocarbon component and a plurality of second responses for the at least one of the plurality of the second chlorofluorocarbon fragment ions per mole of the second chlorofluorocarbon component; and
determining a first measurement signal peak response corresponding to the plurality of the first responses for the at least one of the plurality of the first chlorofluorocarbon fragment ions per mole of the first chlorofluorocarbon component and a second measurement signal peak response corresponding to the plurality of the second responses for the at least one of the plurality of the first chlorofluorocarbon fragment ions per mole of the first chlorofluorocarbon component.

17. The method of claim 16, wherein the selecting the at least one of the plurality of the first mass spectra signals and the at least one of the plurality of the second mass spectra signals comprises determining the first chlorofluorocarbon fragment ion created by the first chlorofluorocarbon component in relative abundance to the second chlorofluorocarbon component and the second chlorofluorocarbon fragment ion created by the second chlorofluorocarbon component in relative abundance to the first chlorofluorocarbon component.

18. The method of claim 16, wherein the first molar response for the first chlorofluorocarbon component relative to the second chlorofluorocarbon component at the at least one of the plurality of the first chlorofluorocarbon fragment ions is greater than the second molar response for the first chlorofluorocarbon component relative to the second chlorofluorocarbon component at the at least one of the plurality of the second chlorofluorocarbon fragment ions.

19. The method of claim 16, wherein the first chlorofluorocarbon component is CFC-113 and the second chlorofluorocarbon component is CFC-113a, wherein the first molar response for CFC-113 relative to CFC-113a at ion 103 is about 5:1, and wherein the second molar response for CFC-113 relative to CFC-113a at ion 117 is about 1:15.

20. The method of claim 16, wherein the plurality of first responses for the at least one of the plurality of the first chlorofluorocarbon fragment ions per mole of the first chlorofluorocarbon component and the plurality of the second responses for the at least one of the plurality of the second chlorofluorocarbon fragment ions per mole of the second chlorofluorocarbon component comprises a first response factor corresponding to the at least one of the plurality of the first chlorofluorocarbon fragment ions per mole of the first chlorofluorocarbon component, a second response factor corresponding to the at least one of the plurality of the first chlorofluorocarbon fragment ions per mole of the second chlorofluorocarbon component, a third response factor corresponding to the at least one of the plurality of the second chlorofluorocarbon fragment ions per mole of the first chlorofluorocarbon component, and a fourth response factor corresponding to the at least one of the plurality of the second chlorofluorocarbon fragment ions per mole of the second chlorofluorocarbon component.
